(19) United States
(12) Patent Application Publication
(10) Pub. No.: US 2026/0146027 A1
(43) Pub. Date: May 28, 2026

(54) REBLASTATIN DERIVATIVE AND ITS USE IN NERVOUS SYSTEM DISEASES

(71) Applicant: CHINESE ACADEMY OF MEDICAL SCIENCES, Institute of Basic Medical Sciences, Beijing (CN)

(72) Inventors: Qi Xu, Beijing (CN); Longze Sha, Beijing (CN); Xiaoming YU, Beijing (CN); Yunfeng Li, Beijing (CN)

(21) Appl. No.: 19/141,714

(22) PCT Filed: Mar. 20, 2025
(86) PCT No.: PCT/CN2025/083743

§ 371 Date: Jun. 20, 2025

(30) Foreign Application Priority Data
Nov. 26, 2024 (CN) 202411701516.9

(51) Int. Cl. C07D 225/06; A61K 31/395; A61K 31/496; A61K 31/69; A61P 25/08; A61P 25/16; A61P 25/28; C07F 5/02

(52) U.S. Cl. CPC C07D 225/06; A61K 31/395; A61K 31/496; A61K 31/69; A61P 25/08; A61P 25/16; A61P 25/28; C07F 5/025

(57) ABSTRACT

A reblastatin derivative and its use in nervous system diseases. By modifying the 18-position hydroxyl of reblastatin, the therapeutic activity of the nervous system can be improved. In addition, especially when it is replaced with a methyl group, the lipid solubility of the compound can be increased, so that the brain-passing efficiency is improved.

### CROSS-REFERENCE TO RELATED APPLICATIONS

This application is a National Stage Application of PCT application No. PCT/CN2025/083743, filed on Mar. 20, 2025, which claims the priority and benefit of Chinese patent applications No. 202411701516.9, filed on Nov. 26, 2024. The entireties of PCT application No. PCT/CN2025/083743 and Chinese patent applications No. 202411701516.9 are hereby incorporated by reference herein and made a part of this specification.

### TECHNICAL FIELD

The present disclosure relates to the technical field of pharmaceutical chemistry, and in particular to a reblastatin derivative and its use in nervous system diseases.

### BACKGROUND ART

Excitotoxicity is a common pathological phenomenon in many brain diseases. It is widely present in diseases such as epilepsy, Alzheimer's disease (AD), Parkinson's disease (PD), and amyotrophic lateral sclerosis (ALS). It is characterized by the accumulation of large amounts of excitatory neurotransmitters dominated by glutamic acid, which cause toxic damage to neurons and trigger a series of neuropathological changes, including: (1) causing abnormal discharges in neurons, which induce epilepsy in extreme cases; (2) overactivating nucleases and proteases in neurons, so that the neurons lose their homeostasis; (3) causing oxidative stress damage to neurons; and (4) directly inducing apoptosis or autophagy of neurons in severe cases.

Excitotoxicity plays an important role in the pathogenesis of many nervous system diseases and is considered to be one of the key factors that destroy normal brain function and cause neurodegenerative lesions. In the past, researchers have focused on the repair strategies that neurons may adopt after suffering excitatory damage: alleviating or reversing the pathological phenotype of the disease through drug or through molecular intervention, cell therapy, and other methods. In recent years, the methods of transplantation of neural stem cells and inhibiting nerve apoptosis have brought hope to the treatment of diseases such as ALS, AD and PD. However, there are also some problems in the above measures. For example, in cell transplantation therapy, the survival rate of stem cells is low and the transplanted neurons are difficult to integrate into the existing neural network.

One hypothesis is that the limited effectiveness of such treatments is due to their failure to block the “strike mechanism” that is always present during the disease process. If it can be started from the attack mechanism of upstream of the disease, it is possible to achieve better therapeutic effect.

Astrocytes can maintain normal glutamic acid level in the neural microenvironment by taking up and releasing glutamic acid. In particular, the glutamate transporter 1 (GLT1, also known as excitatory amino acid transporter 2, EAAT2) plays the most important glutamic acid clearance function. GLT1 is a membrane integral protein specifically expressed by astrocytes. It transports glutamic acid from the extracellular to the intracellular compartment through active transport, so that the glutamic acid concentration in the neural microenvironment is maintained within a normal range. Intracellular glutamic acid is converted into glutamine by glutamine synthetase (GS) and enters the metabolic pathway. GLT1 can also prevent neurons from becoming overexcited by rapidly clearing glutamic acid from receptors on the postsynaptic membrane. Under the action of GLT1, the difference in glutamic acid concentration inside and outside the cell can be as high as 10,000 times. GLT1 is the most important glutamic acid inward transporter in the brain, which is responsible for nearly 80% of glutamic acid clearance.

GLT1 is abnormally downregulated in a variety of nervous system diseases, which results in a severe loss of glutamic acid clearance ability in astrocytes. It may be an important cause of abnormal glutamic acid accumulation. According to the study on patients with refractory temporal lobe epilepsy, both mRNA and protein of GLT1 are downregulated in the patients' epileptogenic focus (sclerotic hippocampal tissue). AD and epilepsy have certain comorbidities. Many studies have confirmed that AD patients also have neuronal damage caused by excitotoxicity in their brains. In addition, a significant decrease in the protein level of GLT1 was found in the autopsy brain tissue of AD patients. According to the study on autopsy brain tissue of ALS patients, the loss of GLT1 protein is as high as 90%. Subsequent functional studies showed that abnormal internalization and protein degradation of GLT1 under pathological conditions may be an important reason for the insufficient amount of protein. In addition, the researchers also observed downregulation of GLT1 in a mouse model of tuberous sclerosis.

Functional studies at animal level provide solid evidence for the relationship between GLT1 loss and disease phenotypes. At first, GLT1 transgenic mice have the ability to resist epileptic seizures, which is manifested in that under the induction of epilepsy-causing drugs (pilocarpine, an agonist of muscarinic acetylcholine receptors, can be used to establish a rodent model of epilepsy through intraperitoneal injection), the death rate of hippocampal neurons in GLT1 transgenic mice is reduced, the degree of mossy fiber sprouting and other pathological phenotypes related to hippocampal sclerosis are alleviated, and the number of spontaneous chronic epileptic seizures in GLT1 transgenic mice is also reduced by about 50%. On the contrary, inhibiting the expression of GLT1 by injecting antisense RNA can lead to a large accumulation of glutamic acid in the brain of mice, which induces neurodegeneration and gradually paralyzes the mice. These studies indicate that astrocytes play an important neuroprotective function through their specifically expressed GLT1 molecule. The loss of GLT1 is sufficient to cause excitotoxic damage to the nervous system.

In summary, abnormal downregulation of GLT1 protein may be an important cause of excessive accumulation of glutamic acid in the brain of patients with epilepsy, AD, ALS, etc. In recent years, some researchers have proposed a hypothesis of excessive degradation of GLT1 protein, and found that HSP90 protein promoted 20S proteasome-dependent GLT1 protein degradation by recruiting GLT1 to the proteasome. In addition, reactive astrogliosis is a pathological phenomenon common to almost all brain diseases, and this process itself is accompanied by increased expression of HSP90 molecules. Therefore, treatment with HSP90 inhibitors can increase the level of GLT1 protein in brain diseases such as temporal lobe epilepsy and AD. In terms of therapeutic effects, in mice with chronic temporal lobe epilepsy induced by kainic acid, two HSP90 inhibitors 17AAG and NVP-HSP990 can relieve epileptic seizures. In the AD mouse model, NVP-HSP990 can reduce epileptic discharges in the brain and alleviate cognitive decline. Since the expression level of HSP90 in astrocytes is much lower than that in neurons, and the overall expression level of HSP90 in the brain is comparable to that in other tissues and organs, the dosage of HSP90 inhibitors used in the treatment of brain diseases is much lower than the severe dose for anti-tumor treatment, but it can increase the level of GLT1 protein and exert anti-epileptic and anti-AD effects. These research results indicate that HSP90 inhibitors at low doses can have therapeutic effects on brain diseases with neuroexcitotoxicity such as epilepsy and AD.

However, existing HSP90 inhibitors have certain problems. For example, both 17AAG and NVP-HSP990 have their own disadvantages: 17AAG has poor drugability (low water solubility, low bioavailability, and toxicity); NVP-990 has optic neurotoxicity in clinical trials; (2) More importantly, HSP90 inhibitors have great limitations in the treatment of brain diseases (such as permeability to the blood-brain barrier). Therefore, it is necessary to further optimize the system towards the goal of “treating brain diseases”.

Reblastatin is a type of aniline mycin, and its structural formula is as follows:

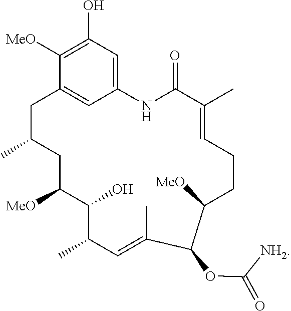 [SMILES: COc1c(O)cc2cc1C[C@@H](C)C[C@H](OC)[C@H](O)[C@@H](C)/C=C(\C)[C@H](OC(N)=O)[C@@H](OC)CC/C=C(\C)C(=O)N2]

Compared with 17AAG, the para-benzoquinone structure on the benzene ring of this compound is replaced by a phenol structure, so its toxicity is reduced. However, its disadvantage is also that its brain-passing efficiency is extremely low, so it is necessary to modify its compound structure to improve its brain-passing efficiency.

### SUMMARY

The object of the present disclosure is to provide a reblastatin derivative and its use in nervous system diseases. By modifying the 18-position hydroxyl of reblastatin, the therapeutic activity of the nervous system can be improved. In addition, especially when it is replaced with a methyl group, the lipid solubility of the compound can be increased, so that the brain-passing efficiency is improved.

In order to achieve the above-mentioned purpose of the disclosure, the technical solution of the present disclosure is as follows:

In the first aspect, the present disclosure provides a use of a reblastatin derivative or a stereoisomer, geometric isomer, tautomer, nitrogen oxide, hydrate, solvate, pharmaceutically acceptable salt or prodrug of the reblastatin derivative in preparing drugs for treating and/or preventing nervous system diseases.

The structural formula of the reblastatin derivative is shown in Formula I:

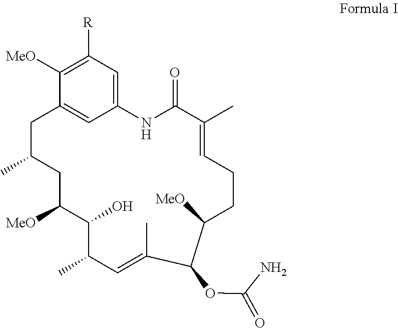

In particular, R is selected from H; halogen; amino; substituted or unsubstituted C1-5 alkyl, C1-5 alkoxy, C3-10 cycloalkyl, C1-5 ester, aryl, amine, heterocyclic group, the substituted substituent is selected from at least one of C1-C5 alkyl, carbonyl, hydroxyl, C1-C5 alkyl, halogen, amine, aryl, or heterocyclic group.

Preferably, the R is selected from substituted or unsubstituted C1-5 alkyl, C1-5 alkoxy, C3-10 cycloalkyl, ester, aryl, amine, heterocyclic group, the substituted substituent is selected from at least one of C1-C5 alkyl, carbonyl, hydroxyl, C1-C5 alkyl, halogen, amine, aryl, or heterocyclic group.

Further preferably, the R is selected from substituted or unsubstituted C1-5 alkyl, C1-5 ester, and heterocyclic group, the substituted substituent is selected from at least one of carbonyl, hydroxyl, C1-C5 alkyl, halogen, amine, aryl, or heterocyclic group, a heteroatom in the heterocyclic group is selected from at least one of N, S, B, or O.

Furthermore preferably, the R is selected from substituted or unsubstituted C1-3 alkyl, C1-5 ester, and heterocyclic group, the substituted substituent is selected from at least one of C1-C5 alkyl, amine, or heterocyclic group, the heteroatom in the heterocyclic group is selected from at least one of N, S, B, or O.

Furthermore preferably, the R is selected from C1-3 alkyl, —OCO—Ra, substituted heterocyclic group, the Ra and the substituted substituent are each independently selected from at least one of C1-C3 alkyl, amino, or heterocyclic group, the heteroatom in the heterocyclic group is selected from at least one of N, S, B, or O.

Furthermore preferably, the R is selected from C1-3 alkyl, —OCO—Ra, substituted heterocyclic group, the Ra is selected from at least one of amino or heterocyclic group, the substituted substituent is selected from C1-C3 alkyl, and the heteroatom in the heterocyclic group is selected from at least one of N, B, or O.

Furthermore preferably, the R is selected from C1-3 alkyl, —OCO—Ra, at least one C1-3 alkyl-substituted pentacyclic heterocyclic group containing B and O, the Ra is selected from at least one of amino or six-membered heterocyclic group containing N.

Furthermore preferably, the R is selected from methyl, —OCO—Ra, at least one C1-3 alkyl-substituted pentacyclic heterocyclic group containing B and O, the Ra is selected from at least one of a C1-3 alkyl-substituted amine, piperazinyl, or C1-3 alkyl-substituted piperazinyl group.

Furthermore preferably, the R is selected from methyl, —OCO—Ra, at least one methyl-substituted pentacyclic heterocyclic group containing B and O, the Ra is selected from at least one of dimethylamino, methylethylamino, diethylamino, piperazinyl, or methyl-substituted piperazinyl group.

Furthermore preferably, the R is selected from methyl, —OCO—Ra,

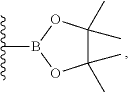 [SMILES: *B1OC(C)(C)C(C)(C)O1]

the Ra is selected from dimethylamino group,

Most preferably, the R is methyl group.

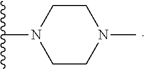 [SMILES: *N1CCN(C)CC1]

Preferably, the nervous system disease is a brain disease.

Preferably, the nervous system disease is selected from at least one of epilepsy, Alzheimer's disease, Parkinson's disease, or amyotrophic lateral sclerosis.

Further preferably, it is at least one of epilepsy or Parkinson's disease.

In a second aspect, the present disclosure provides a reblastatin derivative or a stereoisomer, geometric isomer, tautomer, nitrogen oxide, hydrate, solvate, pharmaceutically acceptable salt or prodrug of the reblastatin derivative. The structural formula is shown in Formula I:

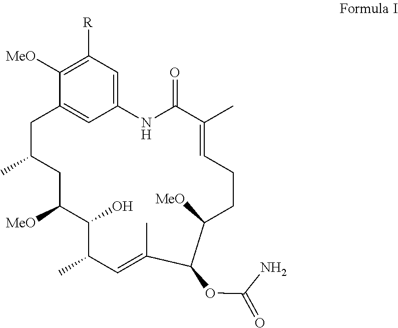

The R is selected from methyl, —OCO—Ra, substituted heterocyclic group, the Ra and the substituted substituent are each independently selected from at least one of C1-C3 alkyl, amino, or heterocyclic group, the heteroatom in the heterocyclic group is selected from at least one of N, S, B, or O.

Preferably, the R is selected from C1-3 alkyl, —OCO—Ra, substituted heterocyclic group, the Ra is selected from at least one of amino or heterocyclic group, the substituted substituent is selected from C1-C3 alkyl, and the heteroatom in the heterocyclic group is selected from at least one of N, B, or O.

Further preferably, the R is selected from C1-3 alkyl, —OCO—Ra, at least one C1-3 alkyl-substituted pentacyclic heterocyclic group containing B and O, the Ra is selected from at least one of amino or six-membered heterocyclic group containing N.

Furthermore preferably, the R is selected from methyl, —OCO—Ra, at least one C1-3 alkyl-substituted pentacyclic heterocyclic group containing B and O, the Ra is selected from at least one of a C1-3 alkyl-substituted amine, piperazinyl, or C1-3 alkyl-substituted piperazinyl group.

Furthermore preferably, the R is selected from methyl, —OCO—Ra, at least one methyl-substituted pentacyclic heterocyclic group containing B and O, the Ra is selected from at least one of dimethylamino, methylethylamino, diethylamino, piperazinyl, or methyl-substituted piperazinyl group.

Furthermore preferably, the R is selected from methyl, —OCO—Ra,

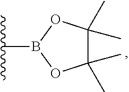 [SMILES: *B1OC(C)(C)C(C)(C)O1]

the Ra is selected from dimethylamino group,

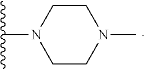 [SMILES: *N1CCN(C)CC1]

Most preferably, the R is methyl group.

In a third aspect, the present disclosure provides a preparation method for the above-mentioned reblastatin derivative, which is selected from the following methods:

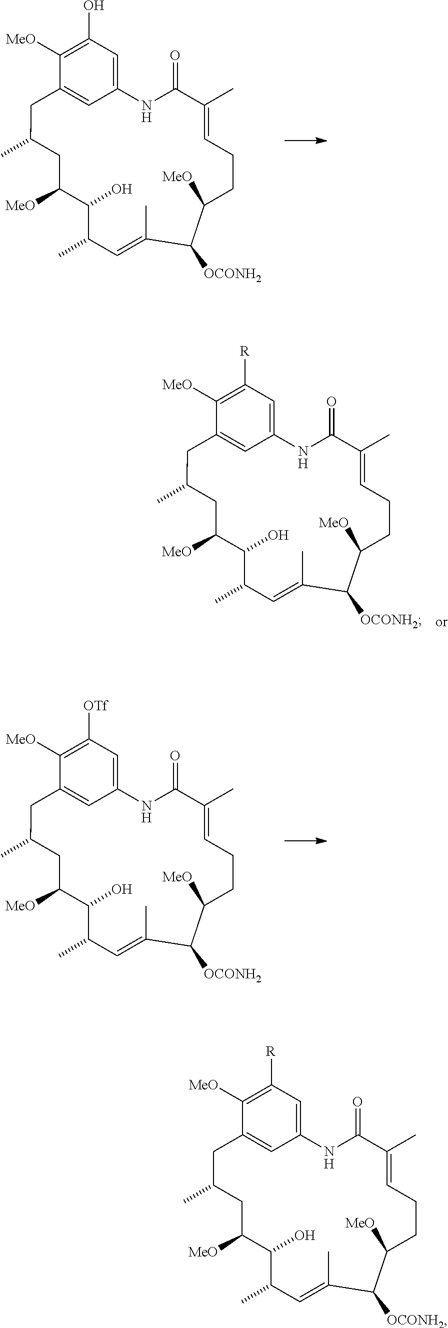

In particular, the R has the same definition as above.

Preferably, when the R is methyl or substituted heterocyclic group, the preparation method is:

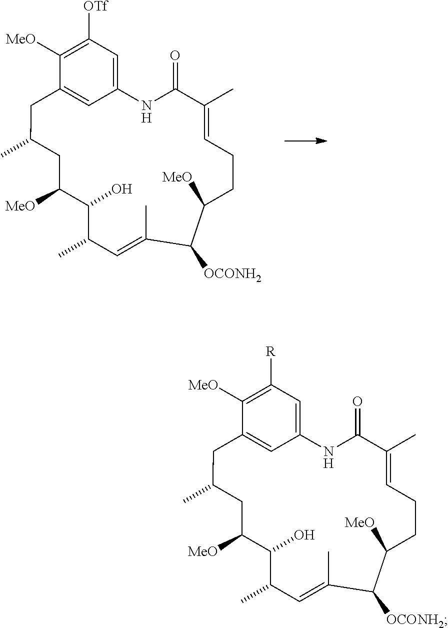

when the R is —OCO—Ra, the preparation method is:

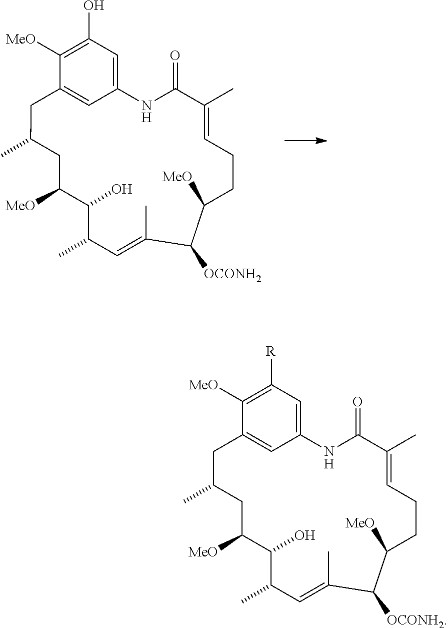

In a fourth aspect, the present disclosure provides a pharmaceutical composition comprising the above-mentioned reblastatin derivative or a stereoisomer, geometric isomer, tautomer, nitrogen oxide, hydrate, solvate, pharmaceutically acceptable salt or prodrug of the reblastatin derivative, and pharmaceutically acceptable carrier or excipient.

The phrase “carrier” is well-known and includes pharmaceutically acceptable materials, compositions or vehicles that are suitable for administering the compounds of the present disclosure to mammals. Carriers include liquid or solid fillers, diluents, excipients, solvents or encapsulating materials, which participate in carrying or transporting the substance of the subject matter from one organ or part of the body to another. Each carrier must be “acceptable” in the sense of being compatible with the other ingredients of the formulation and not injurious to the subject. Some examples of materials that can serve as pharmaceutically acceptable carriers include: sugars, such as lactose, glucose, and sucrose; starches, such as corn starch and potato starch; cellulose and its derivatives, such as sodium carboxymethylcellulose, ethylcellulose, and cellulose acetate; tragacanth, malt, gelatin, and talc; excipients, such as cocoa butter and suppository waxes; oils, such as peanut oil, cottonseed oil, safflower oil, sesame oil, olive oil, corn oil, and soybean oil; diols, such as propylene glycol; polyols, such as glycerol, sorbitol, mannitol, and polyethylene glycol; esters such as ethyl oleate and ethyl laurate, agar; buffers such as magnesium hydroxide and aluminum hydroxide, alginic acid, hot raw water, isotonic saline, ethanol, phosphate buffer, and other nontoxic compatible substances used in pharmaceutical preparations. Wetting agents, emulsifiers and lubricants such as sodium lauryl sulfate and stearic acid esters, as well as coloring agents, release agents, coating agents, sweetening, flavoring and perfuming agents, preservatives and antioxidants may also be present in the composition.

Examples of pharmaceutically acceptable antioxidants include: water-soluble antioxidants, such as ascorbic acid, cysteine hydrochloride, sodium bisulfate, sodium metabisulfite, sodium sulfite, etc.; oil-soluble antioxidants, such as ascorbyl palmitate, butylated benzoic acid (BHA), butylated hydroxytoluene (BHT), lecithin, propyl gallate, α-tocopherol, etc.; and metal mixtures, such as citric acid, ethylenediaminetetraacetic acid (EDTA), sorbitol, tartaric acid, phosphoric acid, etc.

Suitable pharmaceutically acceptable carriers include, but are not limited to, water, saline solutions (e.g., NaCl), alcohol, gum arabic, vegetable oils, benzyl alcohol, polyethylene glycol, gelatin, sugars (e.g., lactose, straight-chain starch or starch), polyethylene glycol, magnesium stearate, talc, silicic acid, viscous paraffin, essential oils, fatty acid esters, carboxymethylcellulose, polyvinylpyrrolidone, and the like. The pharmaceutical compositions can be sterilized and, if desired, mixed with auxiliary agents, such as lubricants, preservatives, stabilizers, wetting agents, emulsifiers, salt buffers for influencing the osmotic pressure, colorants, flavoring and/or aromatic substances, etc., which do not deleteriously react with the active compounds.

The composition may also contain minor amounts of wetting agents, emulsifiers, or pH buffering agents. The composition can be a liquid solution, suspension, emulsion, tablet, pill, capsule, sustained release formulation or powder. The composition can be formulated as a suppository with traditional binders and carriers such as triglycerides. Oral formulation can include standard carriers such as pharmaceutical grades of mannitol, lactose, magnesium stearate, polyvinylpyrrolidone, sodium saccharine, cellulose, magnesium carbonate, and the like.

According to conventional methods, the composition can be formulated into a pharmaceutical composition suitable for intravenous administration to humans. When necessary, the composition may also include a stabilizer and a local anesthetic to ease pain at the site of the injection.

Generally, the ingredients are supplied either separately or mixed together in unit dosage form, for example, as a dry lyophilized powder or water free concentrate in a hermetically sealed container such as an ampoule or sachet indicating the quantity of active agents. When the composition is to be administered by infusion, it can be dispensed with an infusion bottle containing pharmaceutical grade sterile water, saline or dextrose in water. When the composition is administered by injection, an ampoule of sterile water for injection or saline can be provided, so that the ingredients can be mixed prior to administration.

The pharmaceutical compositions of the present disclosure may also include an agent that controls the release of the compound of the present disclosure, thereby providing a timed or sustained release composition.

The pharmaceutical compositions of the present disclosure include those suitable for oral, rectal, topical, vaginal and parenteral (including subcutaneous, intramuscular and intravenous) administration, although the most suitable route in any particular case will depend on the particular subject and the nature and severity of the condition to which the active ingredient is to be administered. The pharmaceutical composition can be prepared by any method known in the field of pharmacy.

The active ingredient can be administered orally in solid dosage forms, such as capsules, tablets, troches, lozenges, granules and powders, or in liquid dosage forms, such as elixirs, syrups, emulsions, dispersions and suspensions. The active ingredient can also be administered parenterally in sterile liquid dosage forms such as dispersions, suspensions or solutions. Other dosage forms in which the active ingredient may be administered are ointments, creams, drops, transdermal patches or powders for topical administration; ophthalmic solutions or suspensions for administration to the eye, i.e., eye drops; sprays or powder compositions for inhalation or intranasal administration, or creams, ointments, sprays or suppositories for rectal or vaginal administration. Gelatin capsules contain the active ingredient and powdered carriers, such as lactose, starch, cellulose derivatives, magnesium stearate, stearic acid, and the like. Similar diluents may be used to prepare compressed tablets. Both tablets and capsules can be formulated as sustained-release products to provide sustained release of the drug over a period of hours. Compressed tablets can be sugar coated or film coated to mask any unpleasant taste and protect the tablet from the atmosphere, or enteric coated for selective disintegration in the gastrointestinal tract. Liquid dosage forms for oral administration may contain coloring and flavoring to increase patient acceptance. In general, water, suitable oil, saline, dextrose (glucose) aqueous solution, and related sugar solutions and glycols such as propylene glycol or polyethylene glycols are suitable carriers for parenteral solutions. Solutions for parenteral administration are preferably water-soluble salt containing the active ingredient, suitable stabilizing agents, and buffer substances as needed. Antioxidants such as sodium bisulfite, sodium sulfite or ascorbic acid, alone or in combination, are suitable stabilizers. Citric acid and its salts and sodium EDTA may also be used. In addition, parenteral solutions may contain preservatives, such as benzalkonium chloride, methyl- or propylparaben, and chlorobutanol.

For administration by inhalation, the compounds of the present disclosure are conveniently delivered in the form of an aerosol spray from pressurized packs or nebulizers. The compounds may also be delivered in powder form for formulation, and the powder composition may be inhaled with the aid of an insufflable powder inhaler device. A preferred delivery system for inhalation is a metered dose inhalation (MDI) aerosol, which may be formulated as a suspension or solution of a compound of formula I in a suitable propellant, for example a fluorocarbon or a hydrocarbon. For ocular administration, ophthalmic preparations can be prepared with a suitable weight percent solution or suspension of a compound of Formula I in a suitable ophthalmic carrier to maintain the compound in contact with the ocular surface for a sufficient time to allow the compound to penetrate the cornea and internal areas of the eye.

Useful pharmaceutical dosage forms for administering the pharmaceutical compositions of the present disclosure include, but are not limited to, hard and soft gelatin capsules, tablets, parenteral injection solutions, and oral suspensions.

In fifth aspect, the present disclosure provides a method for treating a nervous system disease, including administering an effective amount of the above reblastatin derivative or a stereoisomer, geometric isomer, tautomer, nitrogen oxide, hydrate, solvate, pharmaceutically acceptable salt or prodrug of the reblastatin derivative, or the above pharmaceutical composition to an individual. This method can be used in vivo or in vitro. The individual may be a mammal, such as a human.

When the compound of the present disclosure is administered stepwise or in combination with other therapeutic agents, the same dosage forms as described above can be used. When the drugs are administered in physical combination, the dosage form and administration route should be selected based on the compatibility of the combined drugs. The compounds of the present disclosure may be administered as the sole active ingredient or in combination with a second active ingredient, including active ingredients known to be useful in treating the relevant disease.

In some implementations, the amount of the compound is in the range of about 0.001 mg/kg body weight/day to about 1000 mg/kg body weight/day. In other implementations, the amount of the compound ranges from about 0.5 mg/kg body weight/day to about 50 mg/kg body weight/day. In some implementations, the amount of the compound is from about 0.001 g/day to about 7 g/day. In other implementations, the amount of the compound is from about 0.002 g/day to about 6 g/day. In other implementations, the amount of the compound is from about 0.005 g/day to about 5 g/day. In other implementations, the amount of the compound is from about 0.01 g/day to about 5 g/day. In other implementations, the amount of the compound is from about 0.02 g/day to about 5 g/day. In other implementations, the amount of the compound is from about 0.05 g/day to about 2.5 g/day. In other implementations, the amount of the compound is from about 0.1 g/day to about 1 g/day. In other implementations, dosage levels below the lower limit of the aforementioned range may be sufficient. In other implementations, dosage levels above the upper limit of the aforementioned range may be required. In some implementations, the compound is administered in a single dose, once daily. In other implementations, the compound is administered in multiple doses, more than once per day. In some implementations, the compound is administered twice daily. In other implementations, the compound is administered three times per day. In other implementations, the compound is administered four times per day. In other implementations, the compound is administered more than four times per day. In some implementations, the individual to whom the pharmaceutical composition is administered is a mammal. In other implementations, the mammal is a human.

Preferably, the nervous system disease is selected from at least one of epilepsy, Alzheimer's disease, Parkinson's disease, or amyotrophic lateral sclerosis.

Further preferably, it is at least one of epilepsy or Parkinson's disease.

### Interpretation of Terms:

Unless defined otherwise, all technical and scientific terms herein have the same meanings as commonly understood by one of ordinary skill in the art. Unless otherwise indicated, all patents, patent applications, and publications cited herein are incorporated by reference in their entirety. If there are multiple definitions of a term in this document, the definition in this chapter shall prevail.

It is to be understood that both the foregoing general description and the following detailed description are exemplary and explanatory only and are not restrictive of the present disclosure. In the present disclosure, unless otherwise specifically stated, the use of the singular also includes the plural. It should also be noted that the use of “or” means “and/or” unless stated otherwise. Furthermore, the term “include,” as well as other forms, such as “comprise”, “contain” is not limiting.

Definitions of standard chemical terms may be found in reference literature: Carey and Sundberg “ADVANCED ORGANIC CHEMISTRY 4 TH ED.” Vols. A (2000) and B (2001), Plenum Press, New York. Unless otherwise indicated, conventional methods within the skill of the art, such as mass spectroscopy, NMR, IR and UV/Vis spectroscopy and pharmacological methods are employed. Unless specific definitions are set forth, the terms employed herein in the related descriptions of analytical chemistry, synthetic organic chemistry, and pharmaceutical and medicinal chemistry are those known in the art. Standard techniques can be used in chemical syntheses, chemical analyses, pharmaceutical preparation, formulation, and delivery, and treatment of patients. For example, the reaction and purification can be performed using the manufacturer's instructions for using the kit, or according to methods known in the art or the description of the present disclosure. The foregoing techniques and procedures can generally be performed according to conventional methods well known in the art, as described in the various general and more specific references that are cited and discussed throughout the present specification. Throughout the specification, groups and substituents thereof can be selected by one skilled in the art to provide stable moieties and compounds.

When substituents are described by conventional chemical formulas written from left to right, the substituents also include chemically equivalent substituents that would result when the formula is written from right to left. For example, CH2O is equivalent to OCH2.

Unless otherwise stated, general chemical terms such as, but not limited to, “alkyl,” “amine,” “aryl” are used to refer to their optionally substituted forms. For example, “alkyl” as used herein includes optionally substituted alkyl groups.

The term “optional” or “optionally” means that the subsequently described event or circumstance may or may not occur, and that the description includes both the occurrence of said event or circumstance and the non-occurrence of said event or circumstance. For example, according to the definition below, “substituted or unsubstituted” means “unsubstituted” (not substituted by a substituent) or “substituted” (substituted by a substituent) alkyl.

As used herein, C1-n includes C1-2, C1-3, . . . C1-n. For example, the “C1-C5” group means that there are 1 to 5 carbon atoms in the moiety, i.e, the group contains 1 carbon atom, 2 carbon atoms, 3 carbon atoms or 4 carbon atoms. Thus, for example, “C1-4 alkyl” refers to an alkyl group having 1 to 4 carbon atoms, i.e, the alkyl group is selected from methyl, ethyl, propyl, isopropyl, n-butyl, isobutyl, sec-butyl and tert-butyl. Numeric ranges herein, such as “1-10” refer to each integer in the given range, for example “1-10 carbon atoms” means that the group can have 1 carbon atom, 2 carbon atoms, 3 carbon atoms, 4 carbon atoms, 5 carbon atoms, 6 carbon atoms, 7 carbon atoms, 8 carbon atoms, 9 carbon atoms or 10 carbon atoms.

The term “alkyl” as used herein, alone or in combination, refers to an optionally substituted straight chain or optionally substituted branched chain saturated aliphatic hydrocarbon. The “alkyl” herein may preferably have 1 to about 20 carbon atoms, for example 1 to about 10 carbon atoms, or 1 to about 8 carbon atoms, or 1 to about 6 carbon atoms, or 1 to about 4 carbon atoms, or 1 to about 3 carbon atoms. Examples of alkyl herein include, but are not limited to, methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, tert-butyl, n-pentyl, isopentyl, neopentyl, tert-pentyl and hexyl, as well as longer alkyl groups such as heptyl and octyl, etc. When a group defined herein, such as “alkyl” appears in a numerical range, for example, “C1-C6 alkyl” or “C1-6 alkyl” refers to an alkyl that may be composed of 1 carbon atom, 2 carbon atoms, 3 carbon atoms, 4 carbon atoms, 5 carbon atoms or 6 carbon atoms. The alkyl herein also includes the case where no numerical range is specified.

The “alkyl” used in combination herein includes alkyl linked to other groups, such as alkyl in alkoxy, alkyl in alkylthio, hydroxyalkyl, haloalkyl, cyanoalkyl, monoalkylamino, dialkylamino, and the like.

The term “alkoxy” as used herein, alone or in combination, refers to an alkyl ether (O-alkyl). Non-limiting examples of alkoxy include methoxy, ethoxy, n-propoxy, isopropoxy, n-butoxy, isobutoxy, sec-butoxy, tert-butoxy, and the like.

The term “aromatic/aryl” as used herein, alone or in combination, refers to an optionally substituted aromatic hydrocarbon group having 6 to about 20, such as 6-12 or 6-10 ring-forming carbon atoms, which may be a monocyclic aryl, a bicyclic aryl or a multicyclic aryl. The bicyclic aryl or the polycyclic aryl may be a monocyclic aryl fused with other independent rings such as an alicyclic ring, a heterocyclic ring, an aromatic ring, or an aromatic heterocyclic ring. Non-limiting examples of monocyclic aryl include monocyclic aryl with 6 to about 12, 6 to about 10, or 6 to about 8 ring-forming carbon atoms, such as phenyl. Bicyclic aryl includes such as naphthyl. Polycyclic aryl includes such as phenanthrenyl, anthracenyl, and azulenyl.

The term “heteroaryl” as used herein, alone or in combination, refers to an optionally substituted heteroaryl containing from about 5 to about 20, such as 5 to 12 or 5 to 10 backbone ring atoms. In particular, at least one (e.g., 1-4, 1-3, 1-2) of the ring atoms is a heteroatom independently selected from heteroatoms of oxygen, nitrogen, sulfur, phosphorus, silicon, selenium and tin, but not limited thereto. The ring of the group does not contain two adjacent O or S atoms. Heteroaryl includes monocyclic heteroaryl (having one ring), bicyclic heteroaryl (having two rings), or polycyclic heteroaryl (having more than two rings). In implementations where two or more heteroatoms are present in a ring, the two or more heteroatoms may be the same as one another, or some or all of the two or more heteroatoms may be different from one another. The bicyclic heteroaryl or polycyclic heteroaryl may be a monocyclic heteroaryl fused with other independent rings such as an alicyclic ring, a heterocyclic ring, an aromatic ring, or an aromatic heterocyclic ring (collectively referred to as a fused-ring heteroaryl). Non-limiting examples of monocyclic heteroaryl include monocyclic heteroaryl having 5 to about 12, 5 to about 10, 5 to about 7 or 6 backbone ring atoms, for example, non-limiting examples thereof include pyridinyl. Fused ring heteroaryl includes benzimidazolyl, quinolinyl, and acridinyl. Other examples of heteroaryl include, but are not limited to, pyridine, pyrimidine, pyrazine, pyridazine, triazine, furan, thiophene, imidazole, triazole, tetrazole, thiazole, isothiazole, 1,2,4-thiadiazole, pyrrole, pyrazole, oxazole, isoxazole, oxadiazole, benzofuran, benzothiophene, benzothiazole, indole, indazole, quinoline, isoquinoline, purine, carbazole, benzimidazole, pyrrolopyridine, pyrrolopyrimidine, pyrazolopyridine, pyrazolopyrimidine, acridinyl, phenazinyl, benzoxazolyl, benzothiadiazolyl, benzoxazolyl, benzotriazolyl, isoquinolyl, indolizinyl, isothiazolyl, isoindolyl, oxadiazolyl, purinyl, phthalazinyl, pteridinyl, quinazolinyl, quinoxalinyl, triazinyl and thiadiazolyl, and oxides thereof, such as pyridyl N-oxide.

The term “heterocycle” or “heterocyclic group” used herein alone or in combination refers to a non-aromatic heterocycle, which includes a saturated heterocycle or an unsaturated heterocycle (containing an unsaturated bond). One or more (e.g., 1-4, 1-3, 1-2) of the atoms forming the ring are heteroatoms, such as oxygen, nitrogen or sulfur atoms. Heterocycles may include monoheterocycles (having one ring) or biheterocycles (having two bridged rings) or polyheterocycles (having more than two bridged rings). Spirocycles are also included. The heterocyclyl group can have 3 to about 20, such as 3 to about 10, 3 to about 8, 4 to 8, 4 to 7, 5 to about 8, or 5 to about 6 ring-forming atoms. Non-limiting examples of heterocyclic groups include azinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexyl, 3-azabicyclo[4.1.0]heptyl, 3H-indolyl and quinolizinyl, etc. The term also includes all cyclic forms of carbohydrates, including but not limited to monosaccharides, disaccharides, and oligosaccharides. Examples also include, but are not limited to, aziridine, tetrahydrofuran, tetrahydrothiophene, pyrrolidine, oxazolidine, thiazolidine, imidazolidine, isoxazolidine, isothiazolidine, pyrazolidine, morpholine, thiomorpholine, piperazine, piperidinyl, and the like. Heterocyclyl also includes heterocycles having one or more aromatic rings fused (i.e, having a common bond), for example 2,3-dihydrobenzofuran, 1,3-benzodioxolane, benzo-1,4-dioxane, phthalimide, and naphthalimide. The heterocyclic group having one or more aromatic fused groups may be linked to other groups via the aromatic ring or the non-aromatic ring portion. It should be noted that, the heterocyclic group includes substituted heterocyclic groups, that is, other groups can be combined with the heterocyclic ring through heteroatoms or carbon atoms (that is, the heterocyclic ring is connected to the parent molecule or is further substituted).

“Halogen” refers to fluorine, chlorine, bromine or iodine. Fluorine, chlorine and bromine are preferred. A cyano group refers to “—CN”, a hydroxyl group refers to “—OH”, a mercapto group refers to “—SH”, and an amino group refers to “—NH2”.

The term “ester group” has two connection forms, including R1-OCO—R2 and R1-COO—R2. In particular, R1 and R2 refer to the substituents on both sides respectively, and R1 and R2 may be the same or different.

The term “substituted” means that one or more hydrogens on a specified atom are replaced with the specified groups and if the normal valency of the specified atom is not exceeded under existing conditions, the substitution results are in a stable compound.

Unless expressly stated otherwise, all ranges listed herein are inclusive. For example, “the value of n is an integer between 0 and 2” means that n can be 0, 1 or 2.

The term “pharmaceutically acceptable salts” refers to salts prepared from pharmaceutically acceptable non-toxic bases or acids. When the compound of the present disclosure is acidic, its corresponding salt can be easily prepared from an inorganic base or an organic base. Salts derived from such inorganic bases include aluminum, ammonium, calcium, copper (cupric and cuprous), ferric, ferrous, lithium, magnesium, manganese (manganese and manganous), potassium, sodium, zinc salts and the like. Preferred are the ammonium, calcium, magnesium, potassium and sodium salts. Salts prepared from organic bases include primary, secondary, and tertiary amines derived from natural and synthetic sources. Pharmaceutically acceptable organic non-toxic bases that can form salts include arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucosamine, glucosamine, histidine, hydrabamine, isopropylamine, dicyclohexylamine, lysine, methylglucamine, morpholine, piperazine, piperidine, polyamine resins, procaine, purines, theobromine, triethylamine, trimethylamine, tripropylamine, tromethamine, and the like. When the compound of the present disclosure is basic, its corresponding salt can be easily prepared from inorganic or organic acids. Such acids include, for example, acetic acid, benzenesulfonic acid, benzoic acid, camphorsulfonic acid, citric acid, ethanesulfonic acid, fumaric acid, gluconic acid, glutamic acid, hydrobromic acid, hydrochloric acid, isethionic acid, lactic acid, maleic acid, malic acid, mandelic acid, methanesulfonic acid, mucic acid, nitric acid, pamoic acid, pantothenic acid, phosphoric acid, succinic acid, sulfuric acid, tartaric acid, p-toluenesulfonic acid, and the like.

The term “solvate” refers to a complex of variable stoichiometry formed by a solute (ie, a compound of Formula I) or a pharmaceutically acceptable salt thereof and a solvent that does not interfere with the biological activity of the solute. Examples of solvents include, but are not limited to, water, ethanol, and acetic acid. When the solvent is water, the solvate is called a hydrate. Hydrates include, but are not limited to, hemi-, mono-, sesqui-, di-, and tri-hydrates.

The term “prodrug” is a functional derivative of the compounds of the disclosure which is readily convertible in vivo into the desired compound. Various prodrug forms are well known in the art. Referring to discussion on prodrugs provided in Pro-drugs as novel delivery systems (1987) vol. 14 of the A.C.S. Symposium Series by T. Higuchi and V. Stella, Bioreversible Carriers in Drug Design (1987) by Edward B. Roche, ed., American Pharmaceutical Association, and Pergamon Press. The documents: Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, are incorporated herein by reference.

“Subject,” “patient,” or “individual” refers to an individual, including mammals and non-mammals, who has a disease, disorder, condition, or the like. Examples of mammals include, but are not limited to, any member of the class Mammalia: humans, non-human primates (e.g., chimpanzees and other apes and monkeys); livestock, such as cattle, horses, sheep, goats, pigs; domestic animals, such as rabbits, dogs, and cats; laboratory animals, including rodents, such as rats, mice, and guinea pigs, etc. Examples of non-human mammals include, but are not limited to, birds, fish, and the like. In one implementation of the method and composition provided herein, the mammal is a human.

The term “treating and/or preventing” and other similar synonyms include alleviating, alleviating or ameliorating symptoms of a disease or condition, preventing other symptoms, ameliorating or preventing underlying metabolic causes of symptoms, inhibiting a disease or condition, such as arresting the development of a disease or condition, relieving a disease or condition, causing a disease or condition to improve, relieving symptoms caused by a disease or condition, or ceasing symptoms of a disease or condition, and further, the term encompasses the purpose of prevention. The term also includes obtaining a therapeutic effect and/or a prophylactic effect. The therapeutic effect refers to the cure or improvement of the underlying disease being treated. Additionally, cure or amelioration of one or more physiological symptoms associated with the underlying disease is also a therapeutic benefit, such as when an improvement in the patient's condition is observed, even though the patient may still be affected by the underlying disease. For prophylactic benefit, the compositions can be administered to a patient at risk for a particular disease or, even if a diagnosis of the disease has not yet been made, to a patient experiencing one or more of the physiological symptoms of the disease.

As used herein, the terms “effective amount,” “therapeutically effective amount,” or “pharmaceutically effective amount” refer to that amount of at least one agent or compound which, when administered, is sufficient to relieve to some extent one or more of the symptoms of the disease or condition being treated. The result can be reduction and/or alleviation of the signs, symptoms, or causes of a disease, or any other desired alteration of a biological system. For example, an “effective amount” for treatment is the amount of a composition comprising a compound disclosed herein required to provide a clinically significant alleviation of symptoms. The effective amount appropriate for any individual case can be determined using techniques such as dose escalation trials.

As used herein, the terms “taking,” “using,” “administering,” and the like refer to methods capable of delivering a compound or composition to a desired site for biological action. These methods include, but are not limited to, oral routes, intraduodenal routes, parenteral injection (including intravenous, subcutaneous, intraperitoneal, intramuscular, intraarterial injection or infusion), topical and rectal administration. Those skilled in the art are familiar with administration techniques that can be used for the compounds and methods described herein, such as those discussed in Goodman and Gilman, The Pharmacological Basis of Therapeutics, current ed.; Pergamon; and Remington's, Pharmaceutical Sciences (current edition), Mack Publishing Co., Easton, Pa. In a preferred implementation, the compounds and compositions discussed herein are administered orally.

The term “acceptable” with respect to formulation, composition or ingredient, as used herein, means having no long-term detrimental effect on the general health of the subject being treated.

The term “pharmaceutically acceptable” as used herein refers to a substance (such as a carrier or diluent) that does not affect the biological activity or properties of the compounds of the present disclosure and is relatively non-toxic, that is, the substance can be administered to a subject without causing adverse biological reactions or interacting in an adverse manner with any components contained in the composition.

The term “pharmaceutical composition” refers to a biologically active compound optionally mixed with at least one pharmaceutically acceptable chemical component, including but not limited to carriers, stabilizers, diluents, dispersants, suspending agents, thickeners and/or excipients.

The term “carrier” refers to relatively non-toxic chemical compounds or agents that facilitate the introduction of a compound into cells or tissues.

The term “nervous system disease” refers to diseases occurring in the central nervous system, peripheral nervous system, and autonomic nervous system, with sensory, motor, consciousness, and autonomic nervous dysfunction as the main manifestations.

The beneficial effects of the present disclosure are as follows: the present disclosure provides a reblastatin derivative and its use in nervous system diseases, which expands the application of reblastatin derivatives. In addition, for brain nervous system diseases, the present disclosure provides a compound with a new structure, which has excellent brain-passing efficiency, overcomes the problem of low brain-passing efficiency of current drugs, and shows greater advantages in the treatment of brain nervous system diseases.

### BRIEF DESCRIPTION OF THE DRAWINGS

FIG. 1 is a mass spectrum detection diagram of the compound I of the present disclosure.

FIG. 2 is a mass spectrum detection diagram of the compound I-2 of the present disclosure.

FIG. 3 is a mass spectrum detection diagram of the compound I-3 of the present disclosure.

FIG. 4 is a mass spectrum detection diagram of the compound I-4 of the present disclosure.

FIG. 5 is a statistical analysis diagram of IC50 of HepG2 cells after being treated with different concentrations of 18-Me-Reblastatin and 17AAG.

FIG. 6 is a cytotoxicity results diagram of 1-2, 1-3, and I-4.

FIG. 7 is an experimental results diagram of increasing the GLT1 protein level in primary cultured astrocytes.

FIG. 8 is a results diagram of GLT1 detection. The left side shows the Western blotting analysis results of mouse hippocampal tissue homogenate after 18-Me-Reblastatin injection, in which the target protein GLT1 and the internal reference protein Actin are detected respectively. The right side shows the statistical analysis results of the relative level changes of GLT1 protein.

FIG. 9 is a protective effect diagram of the acute epilepsy and the 18-Me-reblastatin induced by PTZ. The data are combined from the results of two animal experiments.

FIG. 10 is a statistical results diagram of epileptic seizure frequency in male APP/PS1 mice.

FIG. 11 is a results diagram of the mouse climbing pole and turning test.

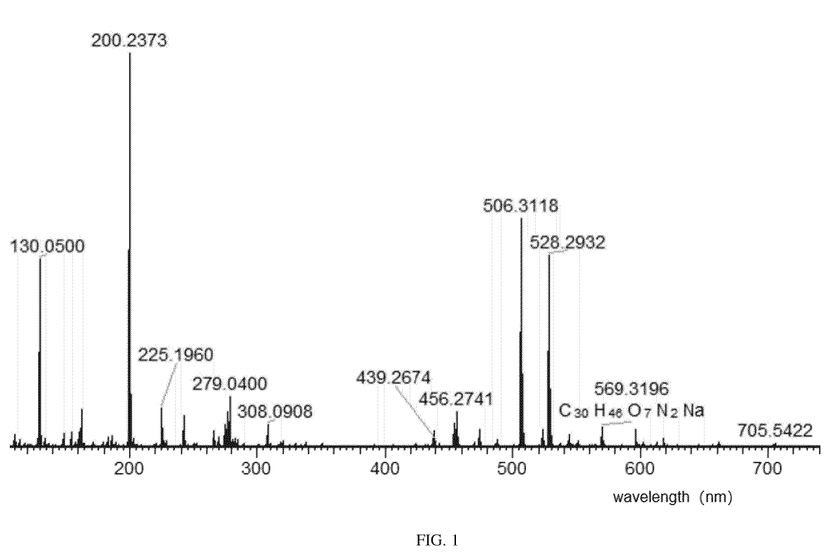

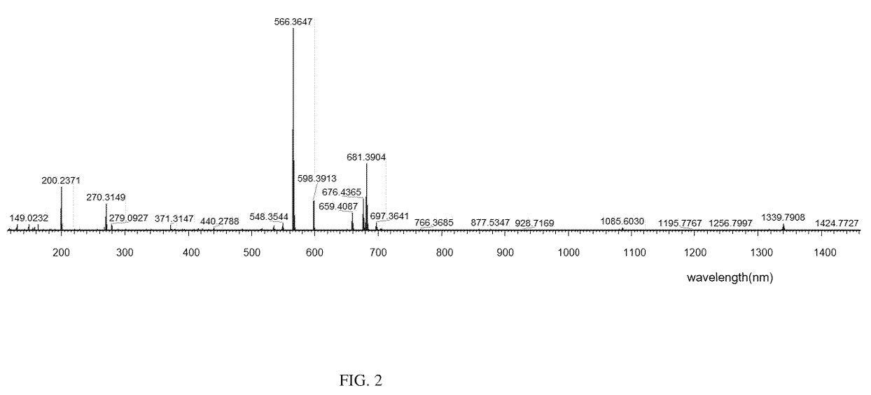

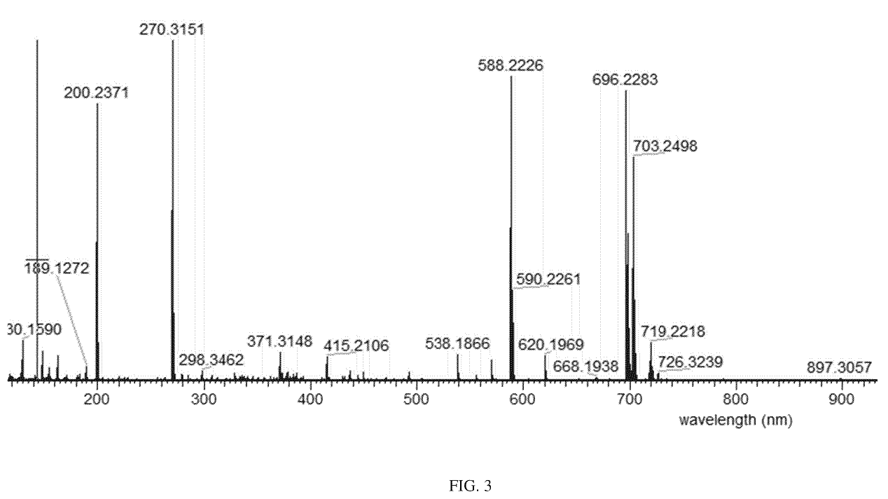

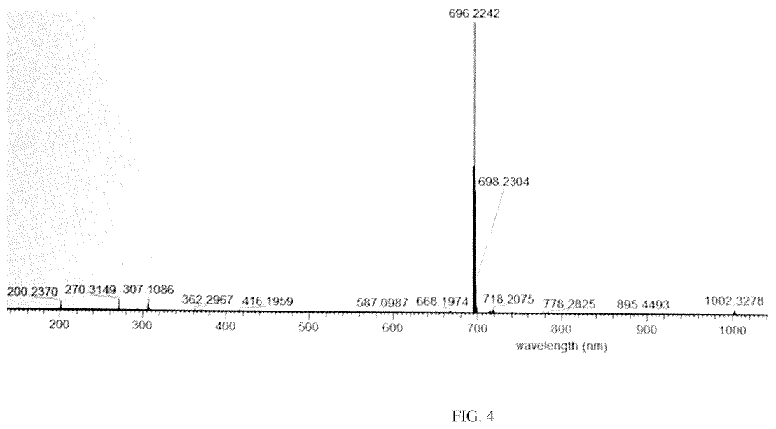

### DETAILED DESCRIPTION

The following non-limiting examples may enable a person skilled in the art to more fully understand the present disclosure, but are not intended to limit the present disclosure in any way. The following contents are merely exemplary of the scope of protection claimed by the present disclosure. Those skilled in the art may make various changes and modifications to the disclosure of the present disclosure based on the disclosed contents, and such changes and modifications should also fall within the scope of protection claimed by this disclosure.

The present disclosure is further described in the form of specific examples. Unless otherwise specified, the various chemical reagents used in the examples of the present disclosure were obtained through conventional commercial channels. Unless otherwise specified, the contents stated below are all by mass. Unless otherwise specified, it is understood that the operation is carried out at room temperature.

The compounds of the present disclosure having the formulae described herein can be prepared according to the procedures illustrated in the following general synthetic schemes from commercially available starting materials or starting materials that can be prepared using literature procedures. The variables (e.g., R) in each general synthetic scheme are as defined herein. It will be noted by those of ordinary skill in the art that in the reaction procedures and synthetic schemes described herein, the order of certain steps may be varied, such as the introduction and removal of protecting groups.

In the present disclosure, 18-Me-Reb and 18-Me-Reblastatin both refer to Compound I.

### Compound Preparation

### Example 1 Preparation of 18-Me-Reblastatin (Compound I)

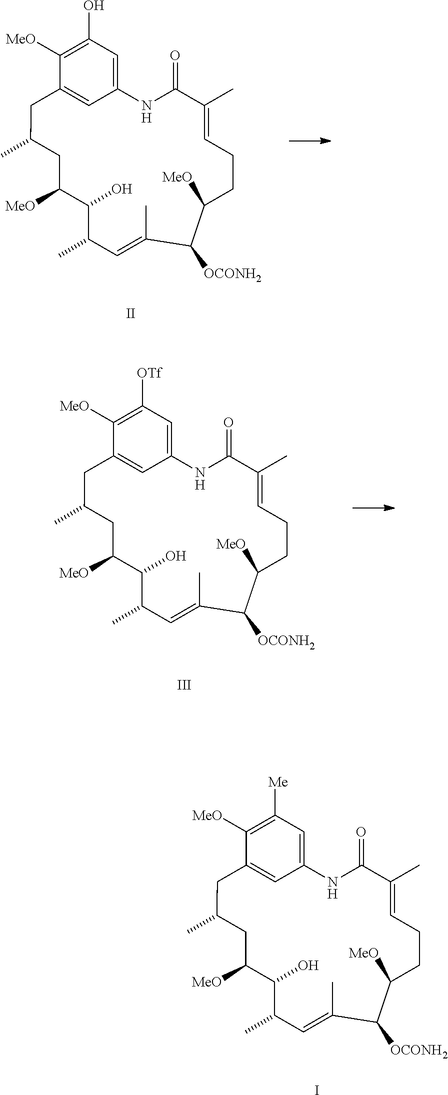

### (1) Preparation of 18-OTf-Reblastatin (Compound III)

Compound II (274.3 mg) was dissolved in anhydrous DMF (2 mL), and DIPEA (0.26 mL), Tf2NPh (268 mg) and DMAP (7 mg) were added. The reaction bottle was then placed in an oil bath at 40° C. for 5 hours. The starting material spot disappeared under TLC monitoring. The reaction solution was cooled to room temperature, quenched with water and extracted with ethyl acetate. The organic phases were combined, washed with saturated brine, dried over anhydrous sodium sulfate, filtered, concentrated under reduced pressure, and purified by column chromatography to obtain compound III (301 mg, yield 88%).

ESI (m/z): 703.72 [M+Na]+

### (2) Preparation of 18-Me-Reblastatin (Compound I)

Compound III (301 mg), K3PO4 (280 mg), and Pd (PPh3) 4 (31 mg) were added to a single-necked round-bottom flask, and the atmosphere was changed to argon protection. Then, dioxane (10 mL) was added, and ultrasonic degassing was performed. Further, a TMB solution in THE (3.5 M, 151 μL) was added, and the reaction flask was placed in an oil bath and heated for reaction (100° C.) for 4 hours. After the reaction was completed as monitored by TLC, the reaction solution was cooled to room temperature, quenched with water, extracted with ethyl acetate. The organic phases were combined, washed with saturated brine, dried over anhydrous sodium sulfate, filtered, concentrated under reduced pressure, and purified by column chromatography to obtain target compound I (200 mg, yield 83%).

ESI (m/z): 569.32 [M+Na]+

### Example 2 Preparation of Compound I-2

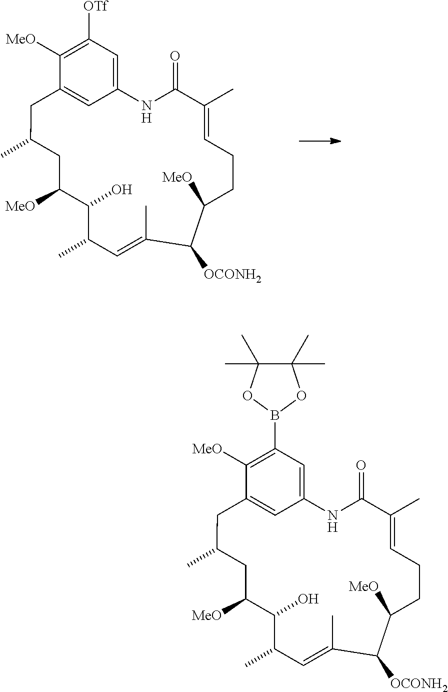

Compound III (14 mg), KOAc (8 mg) and Pd(dppf) 2C12 (2 mg) were added to a round-bottom flask, the atmosphere was changed to argon protection, dioxane (1 mL) was added, ultrasonic degassing was performed, and the reaction solution was placed in an oil bath for heating reaction (100° C.), and the reaction was performed for 1 hour. The reaction solution was cooled to room temperature, diluted with ethyl acetate, and the reaction was quenched with water. The liquids were separated and extracted with ethyl acetate. The organic phases were combined, washed with saturated brine, dried over anhydrous sodium sulfate, filtered, concentrated under reduced pressure, and purified by column chromatography to obtain the target compound I-2 (7 mg, yield 52%).

ESI (m/z): 681.39 [M+Na]+

### Example 3 Preparation of Compound I-3

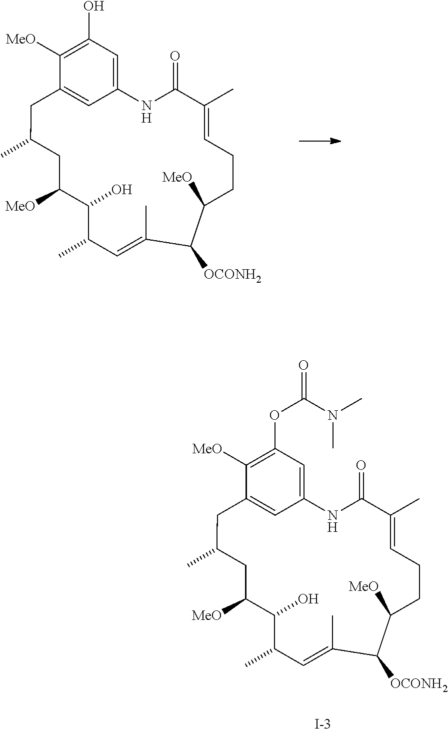

Compound I-3 (20 mg) was added to a round-bottom flask, and the atmosphere was changed to argon protection. DMF (2 mL) was added, and the reaction solution was cooled in an ice-water bath. Potassium carbonate (10 mg) was added, and then acyl chloride (5 μL) was added, and then the temperature was raised to room temperature for reaction for 1 hour. After the reaction was completed, ethyl acetate was added to dilute, water was added and the liquids were separated and extracted with ethyl acetate. The organic phases were combined and washed with saturated brine, dried over anhydrous sodium sulfate, filtered, concentrated under reduced pressure, and purified by column chromatography to obtain the target compound I-3 (8 mg, yield 35%), and some raw materials (10 mg, recovery rate 50%) were recovered.

ESI (m/z): 620.20 [M+Na]+

### Example 4 Preparation of Compound I-4

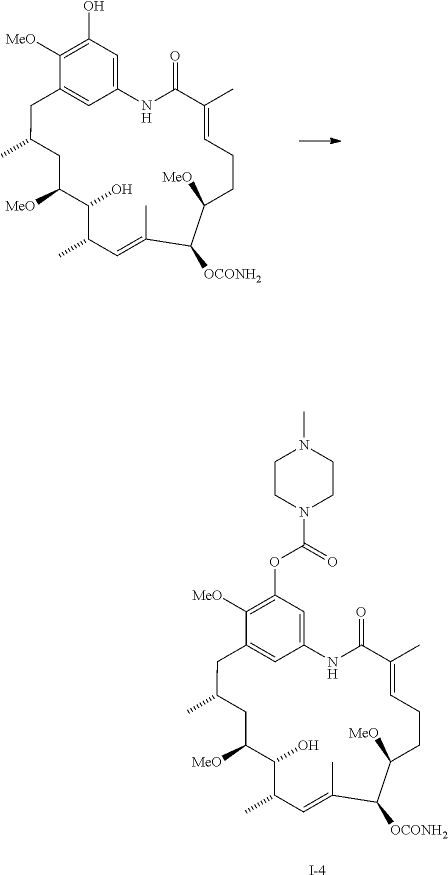

Compound I-3 (5 mg) was added to a round-bottom flask, and the atmosphere was changed to argon protection. DMF (0.5 mL) was added, and the reaction solution was cooled in an ice-water bath. Potassium carbonate (3 mg) was added, and then acyl chloride (2 mg) was added,
and then the temperature was raised to room temperature for reaction for 1 hour. After the reaction was completed, ethyl acetate was added to dilute, water was added and the liquids were separated and extracted with ethyl acetate. The organic phases were combined and washed with saturated brine, dried over anhydrous sodium sulfate, filtered, concentrated under reduced pressure, and purified by column chromatography to obtain the target compound I-4 (4 mg, yield 65%).

ESI (m/z): 698.23 [M+Na]+

### Effect Detection

### 1. Cytotoxicity Test

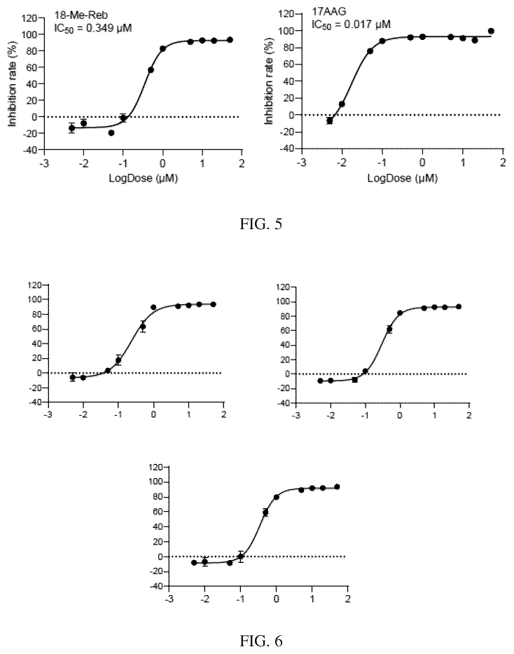

HepG2 cells were used to carry out CTG (CELL TITER-GLO) luminescence method to detect cell proliferation inhibition rate. Screening experiment of four compounds. The compound screening concentrations were 0.005, 0.01, 0.05, 0.1, 0.5, 1, 5, 10, 20, and 50 μM, respectively. The cells were cultured in a cell culture incubator at 37° C., 5% CO2 for 2 days, and then CTG detection was carried out. The IC50 was calculated and the results are shown in FIG. 5. It can be seen that the IC50 of 17AAG is 0.017 μM and the toxicity of 18-Me-Reb is 0.349 μM, which is much lower than the toxicity of 17AAG (19.5 times).

In addition, we also evaluated the cytotoxicity of I-2, I-3, and I-4. As shown in FIG. 6, the IC50 values are 0.301 μM, 0.238 μM, and 0.347 μM, respectively. The results were similar to those of 18-Me-Reb and were much lower than the toxicity of 17AAG.

### 2. Experiment on Increasing the Level of GLT1 Protein in Primary Cultured Astrocytes

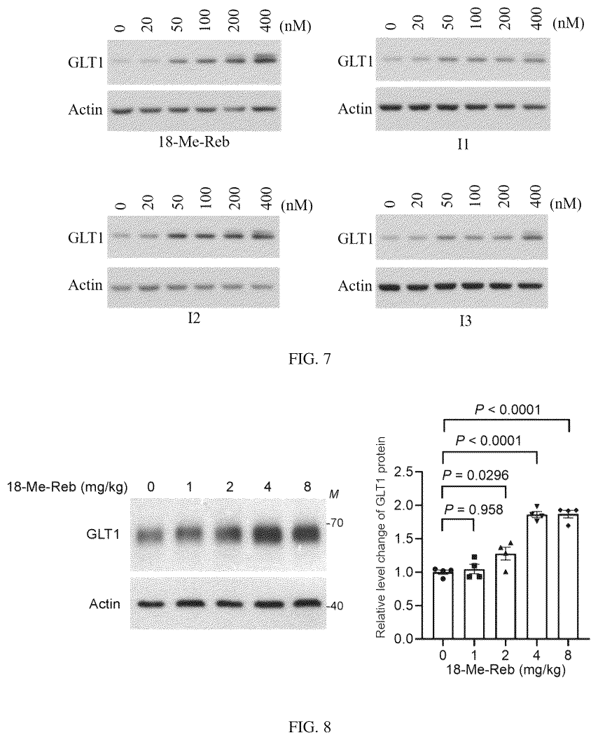

In order to detect whether the above four compounds can increase the expression level of GLT1, primary astrocytes were cultured and the above four compounds were treated on day 14 of culture. The treatment doses were 0, 20, 50, 100, 200, and 400 nM, respectively. After 48 h of compound treatment, astrocytes were collected for Western blotting detection of the levels of GLT1 and Actin proteins. The results are shown in FIG. 7, which show that all four compounds can increase the level of GLT1 protein.

### 2. Experiment on Increasing GLT1 Protein Level in Mouse Hippocampal Tissue by 18-Me-Reblastatin

Twenty male and female C57/BL6j mice aged 9 weeks were used, with 4 mice in each dose group (half male and half female). The mice were injected intraperitoneally with doses of 0, 1, 2, 4, and 8 mg/kg. After injection on the 1st, 3rd, and 5th days, the hippocampal brain tissue of the mice was obtained on the morning of the 6th day for tissue homogenization, and 10 μl (20 μg) of protein was taken for Western blotting analysis. The statistical method was One-way ANOVA. As shown in FIG. 8, the results show that the protein level of GLT1 gradually increased in a dose-dependent manner and reached a maximum at 4 mg/kg injection dose.

### 3. Blood-Brain Barrier Permeability Test

### 3.1 Blood-Brain Barrier Permeability Test of Compound I

At first, the blood drug concentration of mice was tested after once intraperitoneal injection at a dose of 4 mg/kg, and the relevant pharmacokinetic parameters and the brain-blood ratio of the compound were calculated. The peak time of blood drug concentration after injection is 0.14 hours, and the half-life (T1/2) of the compound is 2.07 hours. By testing the compound concentrations in plasma and brain at 0.083, 0.167 and 0.5 hours after injection respectively, the brain-to-blood ratios at these three time points were found to be 0.35, 0.52 and 0.54. This means that about half of the compound can pass through the brain and achieve the properties of a central nervous system drug.

TABLE 1
Blood drug concentration detection at different time points
after once intraperitoneal injection of 18-Me-Reblastatin

| Time | 18-Me-Blood drug concentration (ng/ml) | | | | | CV |
|---|---|---|---|---|---|---|
| (h) | 101 | 102 | 103 | Mean | SD | (%) |
| 0.083 | 22.44 | 24.19 | 17.88 | 21.50 | 3.26 | 15.16 |
| 0.167 | 25.34 | 17.81 | 27.33 | 23.49 | 5.02 | 21.37 |
| 0.5 | 11.94 | 9.80 | 10.74 | 10.82 | 1.07 | 9.92 |
| 1 | 9.45 | 5.53 | 6.37 | 7.12 | 2.06 | 28.96 |
| 2 | 6.26 | 4.47 | 5.54 | 5.42 | 0.90 | 16.60 |
| 4 | 3.24 | 1.30 | 3.21 | 2.58 | 1.11 | 43.11 |
| 8 | BLQ | BLQ | BLQ | NA | NA | NA |
| 12 | BLQ | BLQ | BLQ | NA | NA | NA |
| 24 | BLQ | BLQ | BLQ | NA | NA | NA |

In Table 1, Mean: mean value, SD: standard deviation, CV: coefficient of variation, BLQ: below limit of quantitation (not detected), NA: not available.

TABLE 2
Pharmacokinetic parameters of 18-Me-Reblastatin

| | | | HL- | | Cₘₐₓ | | AUCINF-obs | | |
|---|---|---|---|---|---|---|---|---|---|
| | | | Lambda-Z | Tₘₐₓ | Cₘₐₓ | AUClast | AUC_{(0-∞)} | MRTlast | MRTINF-obs |
| Animal | Route of | Dose | T_{1/2} | Tₘₐₓ | ng/mL | AUC₍₀₋ₜ₎ | h*ng/mL | MRT₍₀₋ₜ₎ | MRT_{(0-∞)} |
| No. | administration | mg/kg | h | h | (g) | h*ng/mL(g) | (g) | h | h |
| 101 | IP | 4 | 1.96 | 0.17 | 25.34 | 31.85 | 41.03 | 1.33 | 2.56 |
| 102 | IP | 4 | 1.28 | 0.08 | 24.19 | 21.97 | 24.37 | 1.16 | 1.63 |
| 103 | IP | 4 | 2.95 | 0.17 | 27.33 | 27.96 | 41.63 | 1.34 | 3.61 |
| Mean | | | 2.07 | 0.14 | 25.62 | 27.26 | 35.68 | 1.28 | 2.60 |
| SD | | | 0.84 | 0.05 | 1.59 | 4.98 | 9.80 | 0.10 | 0.99 |

In Table 2, IP: intraperitoneal injection, Mean: mean value, SD: standard deviation, HL-Lambda-Z: half-life, AUC: area under the concentration-time curve, representing the bioavailability of the drug (the extent to which the drug is absorbed and utilized in the human body), the larger the AUC, the higher the bioavailability, and vice versa, AUC(0-t): AUC from time 0 to the last quantifiable time point, AUC(0-∞): AUC from time 0 to infinity, Tmax: time when the drug concentration reaches peak, Cmax: peak concentration of the drug, AUClast: AUC from the start of administration to the last point, AUCINF-obs: AUC from the start of administration to the theoretically extrapolated infinity, MRTlast: average residence time from the administration to the last quantifiable concentration time, MRTINF-obs: average residence time of the drug from the start of administration to the theoretically extrapolated infinity, T1/2: drug half-life, MRT(0-∞): average residence time of the drug from time 0 to the last quantifiable time

TABLE 3
Detection of brain concentration 0.083 hours after
one intraperitoneal injection of 18-Me-Reblastatin

| Time | 18-Me-Brain tissue concentration (ng/g*) | | | | | |
|---|---|---|---|---|---|---|
| (h) | 201 | 202 | 203 | Mean | SD | CV(%) |
| 0.083 | 7.80 | 7.72 | 6.98 | 7.50 | 0.45 | 6.00 |

TABLE 4
Cerebral blood ratio 0.083 hours after one intraperitoneal
injection of 18-Me-Rebastatin

| | Brain tissue/Plasma ratio | | | | | |
|---|---|---|---|---|---|---|
| Time (h) | 201 | 202 | 203 | Mean | SD | CV(%) |
| 0.083 | 0.36 | 0.36 | 0.32 | 0.35 | 0.02 | 6.00 |

TABLE 5
Detection of brain concentration 0.167 hours after
one intraperitoneal injection of 18-Me-Reblastatin

| Time | 18-Me-Brain tissue concentration (ng/g*) | | | | | |
|---|---|---|---|---|---|---|
| (h) | 301 | 302 | 303 | Mean | SD | CV(%) |
| 0.167 | 17.19 | 8.96 | 10.41 | 12.19 | 4.39 | 36.03 |

TABLE 6
Cerebral blood ratio 0.167 hours after one intraperitoneal
injection of 18-Me-Rebastatin

| | Brain tissue/Plasma ratio | | | | | |
|---|---|---|---|---|---|---|
| Time (h) | 301 | 302 | 303 | Mean | SD | CV(%) |
| 0.167 | 0.73 | 0.38 | 0.44 | 0.52 | 0.19 | 36.03 |

TABLE 7
Detection of brain concentration 0.5 hours after one
intraperitoneal injection of 18-Me-Reblastatin

| Time | 18-Me-Brain tissue concentration (ng/g*) | | | | | |
|---|---|---|---|---|---|---|
| (h) | 401 | 402 | 403 | Mean | SD | CV(%) |
| 0.5 | 6.70 | 5.73 | 5.09 | 5.84 | 0.81 | 13.90 |

TABLE 8
Cerebral blood ratio 0.5 hours after one intraperitoneal
injection of 18-Me-Rebastatin

| | Brain tissue/Plasma ratio | | | | | |
|---|---|---|---|---|---|---|
| Time (h) | 401 | 402 | 403 | Mean | SD | CV(%) |
| 0.5 | 0.62 | 0.63 | 0.47 | 0.54 | 0.07 | 13.90 |

In Tables 3-8, Mean: mean value, SD: standard deviation, CV: coefficient of variation.

### 3.2 Blood-Brain Barrier Permeability Test of Reblastatin

The plasma and brain tissue concentrations of mice after intraperitoneal injection of Reblastatin were detected according to the above methods, and it was found that Reblastatin was almost undetectable in the brain. No concentration of the compound was detected in the brain 0.5 hours after intraperitoneal injection, indicating that its brain-passing efficiency was low.

TABLE 9
Blood drug concentration and brain tissue concentration detection of Reblastatin

| | Drug | | Sampling | Concentration | | | | | |
|---|---|---|---|---|---|---|---|---|---|
| Dose | administration | | Time | (ng/mL) | | | Mean | | |
| mg/kg | Route | Sample | (h) | 1 | 2 | 3 | (ng/mL) | SD | CV(%) |
| 4 | IP | Plasma | 0.00 | 0.00 | 0.00 | 0.00 | 0.00 | 0.00 | NA |
| | | Plasma | 0.25 | 838.00 | 981.00 | 695.00 | 838.00 | 143.00 | 17.06 |
| | | Plasma | 0.50 | 656.00 | 545.00 | 620.00 | 607.00 | 56.60 | 9.32 |
| | | Brain | 0.50 | BLQ | BLQ | BLQ | — | — | — |
| | | tissue | | | | | | | |

In Table 9, IP: intraperitoneal injection, Mean: mean value, SD: standard deviation, CV: coefficient of variation, BLQ: below limit of quantitation (not detected), NA: not available.

### 4. Protective Effect of Compound I in Acute Epilepsy Induced by Pentetrazodone (PTZ)

PTZ is a central nervous system stimulant that induces seizures primarily by acting as a noncompetitive antagonist at GABA receptors. This inhibition reduces GABA-mediated inhibitory neurotransmission, leading to an imbalance between excitatory and inhibitory signaling in the brain. As a result, neuronal excitability increases, inducing acute epileptic seizures. The PTZ-induced acute epilepsy model is often used for the rapid screening of anti-epileptic and anti-neuroexcitatory compounds.

Nine-week-old male C57/BL6j mice were used. There were 18 mice in the control group and 15 mice in the 18-Me-Reblastatin group. On days 1, 3, and 5 of the experiment, 4 mg/kg of the compound dissolved in DMSO or the solvent control group were injected intraperitoneally in a volume of 100 μl. At noon on the 6th day, PTZ was injected intraperitoneally at a dose of 55 mg/kg to induce epileptic seizures. The time of onset of the first epileptic seizure after PTZ injection and the Racine scale of the maximum level of epileptic seizure were detected.

### Racine Rating Standards:

0) no seizure behavior;
1) the behavior stops suddenly and stares motionless;
2) whisker tremors and/or facial and neck twitch;
3) sitting clonus seizures;
4) tonic-clonic seizures (prone);
5) tonic-clonic seizures (lying on one side, loss of postural control) or violent jumps.

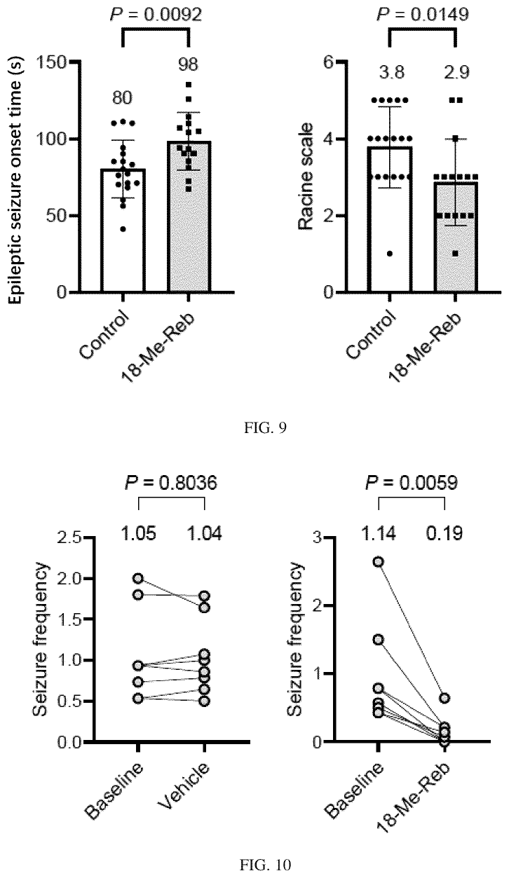

The statistical method was Student's t test, and the results are shown in FIG. 9. From the statistical results, it can be seen that the average onset time of epileptic seizures in the solvent control group was 80 seconds, while the average onset time of epileptic seizures in the 18-Me-Reblastatin treatment group was 98 seconds. Regarding the Racine scale, the average score of the solvent control group was 3.8 points, while the average score of the 18-Me-Reblastatin-treated group was 2.9 points. In conclusion, pretreatment with 18-Me-Reblastatin prolonged the onset time of PTZ-induced epileptic seizures and reduced the severity of seizures. This indicates that 18-Me-Reblastatin has a protective effect on acute epilepsy induced by PTZ.

### 5. Anti-Epileptic and Cognitive Improvement Effects of Compound I in AD Mouse Model (APP/PS1)

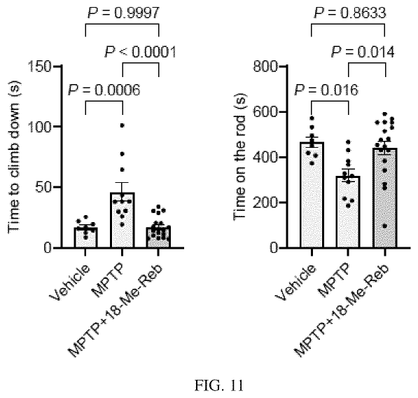

Eight-month-old male APP/PS1 (carrying both APP695swe/PS1-dE9 mutations; Jacksonlab strain number 034832) mice were used. In the EEG test, there were 8 mice in each of the control group and the experimental group. After the electrodes were implanted, the baseline period EEG recordings were at first monitored continuously for 24 h×14 days. From the 15th day, the solvent control group or 18-Me-Reblastatin (4 mg/kg) was intraperitoneally injected once every other day for 7 consecutive times. The EEG recording was at last terminated on day 14 after the first injection. The effects of the compounds were analyzed by comparing the baseline period and post-drug seizure patterns. As shown in FIG. 10, the results showed that the solvent (DMSO) control group had no significant effect on the frequency of epileptic seizures, which was 1.08 times/day in the baseline period and 1.11 times/day after solvent injection. In the experimental group, the baseline frequency of epileptic seizures was 0.96 times/day, and after 18-Me-Reblastatin treatment it was 0.16 times/day, with an average decrease of 83% in the frequency of epileptic seizures.

### 6. Protective Effect of Compound I in MPTP-Induced PD Model

In order to test the therapeutic effect on Parkinson's mouse model, 37 8-week-old male c57 mice were selected and randomly divided into three groups: control group (n=8), MPTP modeling group (n=11) and 18Me administration group (n=18). The administration group was given 18-Me-Reblastatin (4 mg/kg, i.p.) twice in advance, and then the MPTP subacute PD model was established. MPTP was intraperitoneally injected into mice at a dose of 30 mg/kg, and the control group was injected with an equal amount of normal saline once a day for a total of 14 days. During this period, 18-Me-Reblastatin (4 mg/kg) was injected intraperitoneally into the mice in the administration group once every other day, and the control group and the modeling group were injected with an equal amount of DMSO. Behavioral testing was performed two weeks later.

Test 1: Pole climbing experiment. Motor coordination of mice was assessed by observing their ability to grasp and descend a vertical rod. The experimental device consists of a wooden pole with a diameter of 1 cm and a length of 50 cm, with a wooden ball with a diameter of 2.5 cm fixed on the top. The vertical wooden pole was wrapped with gauze to prevent the mice from slipping. During the test, the wooden pole was held at a 900 angle to the ground. The mouse was placed on the top wooden ball, and the time it took for the mouse to spontaneously climb from the top to the bottom was recorded. Each mouse was tested three times, with at least 30 min between experiments, and the three results were averaged.

Test 2: Rotarod test. It was used to assess the coordination and balance abilities of mice. Before all experiments, each mouse was pre-trained for three days, with the speed set at 8 rpm, 12 rpm, and 16 rpm for 5 min. During the formal experiment, the rotarod was set to a uniform acceleration process from 4 rpm to 40 rpm for 10 min, and the time when the mouse fell from the rotation rod was recorded. Each mouse was tested three times, with at least 30 min between experiments, and the three results were averaged.

The results are shown in FIG. 11, which show that 18-Me-Reblastatin can significantly alleviate the sports injury caused by MPTP. It is concluded that 18-Me-Reblastatin also has a protective effect on PD.

It should be noted that the above content is only used to illustrate the present disclosure, rather than to limit the scope of protection of the present disclosure. Simple modifications or equivalent substitutions of the present disclosure by ordinary skilled in the art do not deviate from the essence and scope of the present disclosure.

1.-14. (canceled)

15. A reblastatin derivative or a pharmaceutically acceptable salt of the reblastatin derivative, and a structural formula of the reblastatin derivative is shown in Formula I: 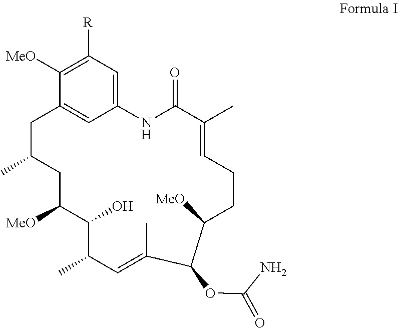
wherein, the R is selected from methyl, —OCO—Ra, 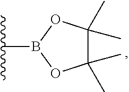 [SMILES: *B1OC(C)(C)C(C)(C)O1]
the Ra is selected from dimethylamino group,

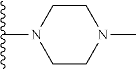 [SMILES: *N1CCN(C)CC1]

16.-20. (canceled)

21. The reblastatin derivative the pharmaceutically acceptable salt of the reblastatin derivative according to claim 15, wherein the R is methyl group.

22. A preparation method for the reblastatin derivative according to claim 15, comprising following steps: 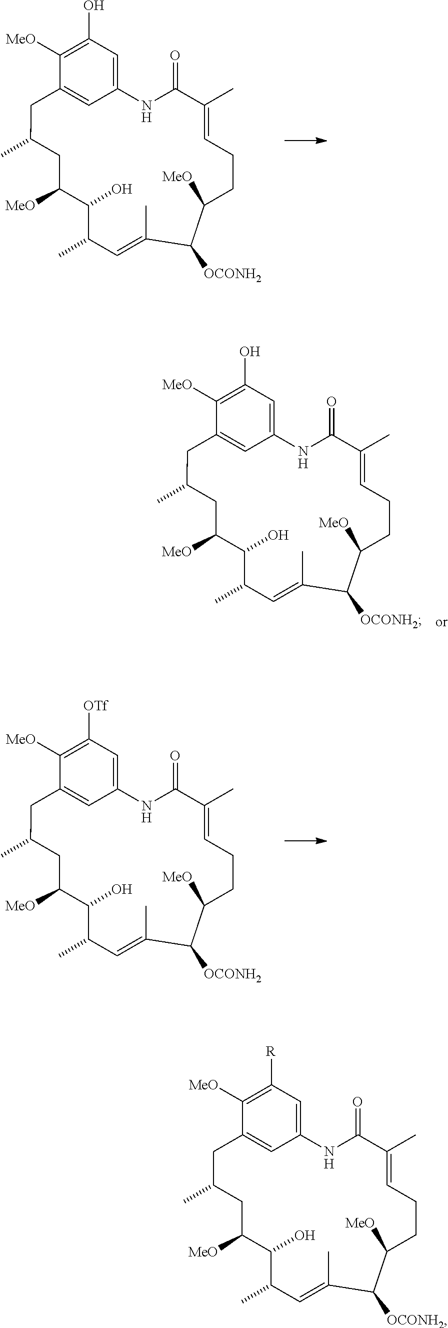
wherein the R is selected from methyl, —OCO—Ra, 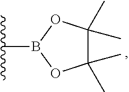 [SMILES: *B1OC(C)(C)C(C)(C)O1]
the Ra is selected from dimethylamino group,

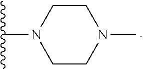 [SMILES: *N1CCN(C)CC1]

23. The preparation method according to claim 22, wherein when the R is the methyl or the substituted heterocyclic group, the preparation method is: 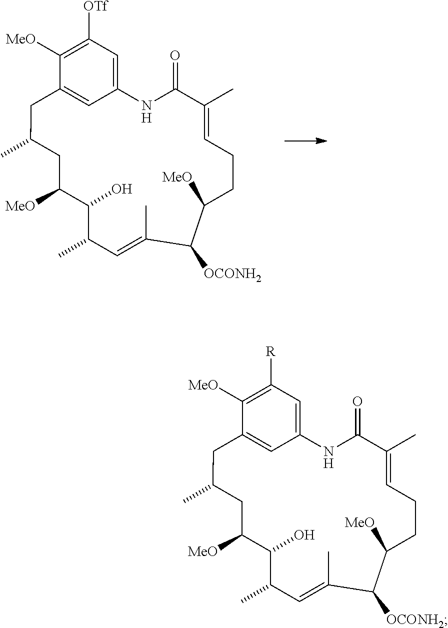
and
when the R is the —OCO—Ra, the preparation method is:

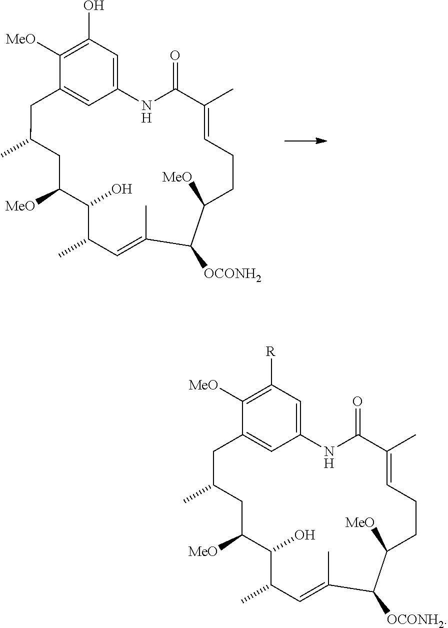

24. A pharmaceutical composition, comprising the reblastatin derivative or the pharmaceutically acceptable salt of the reblastatin derivative according to claim 15, and a pharmaceutically acceptable carrier or an excipient.

25. Use of the reblastatin derivative or the pharmaceutically acceptable salt of the reblastatin derivative according to claim 15 in preparing drugs for treating and/or preventing nervous system disease.

26. The use according to claim 25, wherein the nervous system disease is a brain disease.

27. The use according to claim 25, wherein the nervous system disease is selected from at least one of epilepsy, Alzheimer's disease, Parkinson's disease, or amyotrophic lateral sclerosis.

28. The use according to claim 27, wherein the nervous system disease is selected from at least one of epilepsy or Parkinson's disease.
